(19) United States
(12) Patent Application Publication
(10) Pub. No.: US 2009/0046247 A1
(43) Pub. Date: Feb. 19, 2009

(54) VISION RECOVERY TRAINING DEVICE

(75) Inventors: Eiichi Ookawa, Kanagawa (JP); Hidenori Horie, Kanagawa (JP)

Correspondence Address: PEARNE & GORDON LLP, 1801 EAST 9TH STREET, SUITE 1200, CLEVELAND, OH, 44114-3108, US

(73) Assignees: MATSUSHITA ELECTRIC INDUSTRIAL CO., LTD., Osaka (JP); TECHNOMASTER CO., LTD., Kanagawa (JP)

(21) Appl. No.: 12/088,069

(22) PCT Filed: Sep. 29, 2006
(86) PCT No.: PCT/JP2006/319581

§ 371 Date: Mar. 25, 2008

(30) Foreign Application Priority Data
Sep. 29, 2005 (JP) 2005-284152

(51) Int. Cl. A61B 3/00

(52) U.S. Cl. 351/203

(57) ABSTRACT

It is an object of the present invention to provide a vision recovery training device that can carry out both the training of both eyes and the training of one eye.

The vision recovery training device of the present invention includes a target 2 to be watched; a driving device 3 that moves the target 2 and a visual field plate 13 having three peep windows 5a to 5c through which the target 2 is peeped; and is characterized in that the three peep windows are respectively horizontally arranged in parallel, the peep window 5b provided at a center is located on an extended line of a moving direction of the target 2, other two peep windows 5a and 5c are located at positions spaced by an equal distance from the peep window 5b located at the center and a distance between the other two peep windows 5a and 5c are substantially equal to an interval of pupils of a person.

### TECHNICAL FIELD

The present invention relates to a vision recovery training device for training an image formation accommodative function of an eye.

The summary and a cross-section of a usual vision recovery training device are shown in FIGS. 5 and 6.

As shown in FIG. 5, the vision recovery training device includes a peep portion 111 of one eye and an eye cup 112. Thus, an image dispelled from the peep portion 111 is stared at so that only the one eye can be trained (for instance, see Patent Document 1). Further, as shown in FIG. 6, a device includes a target 113 and a driving device. The target 113 is reciprocated for a prescribed section and the target 113 is stared at so that only both eyes can be trained (for instance, see Patent Document 2).

Patent Document 1: JP-A-2004-201763 (page 6, FIG. 1)
Patent Document 2: JP-A-52-131688 (page 4, FIG. 2)

### DISCLOSURE OF THE INVENTION

### Problems that the Invention is to Solve

However, in the usual vision recovery training device, a problem has arisen that both the training of both eyes and the training of one eye cannot be carried out. When an accommodation ability of a visual acuity is trained, the visual acuity needs to be trained under a state each eye is not allowed to converge. On the other hand, when the visual acuity of both the eyes is trained, the visual acuity needs to be trained under a state including a convergence. The visual acuity needs to be trained under a state that a single target or a target appearing to be a single target is watched by both eyes.

The present invention is devised by solving usual problems and it is an object of the present invention to provide a vision recovery training device that can carry out both the training of both eyes and the training of one eye.

### Means for Solving the Problems

A vision recovery training device of the present invention includes a target to be watched; a driving device that moves the target and a visual field plate having three peep windows through which the target is peeped; and wherein: the three peep windows are provided and respectively horizontally arranged in parallel, the peep window provided at a center is located on an extended line of a moving direction of the target, other two peep windows are located at positions spaced by an equal distance from the peep window located at the center and a distance between the other two peep windows is substantially equal to an interval of pupils of a person.

According to this configuration, the peep hole provided at the center is watched by an eye to be trained so that the training of a single eye can be efficiently carried out. When the training of both eyes is carried out, a person may peep from the peep holes at both ends by both the eyes. Thus, the training of the one eye and the training of both the eyes can be carried out by single device.

Further, the vision recovery training device of the present invention further comprises: a shield plate that covers the visual field plate and is held so as to slide, and wherein two holes are formed on the shield plate and a distance between the two holes formed on the shield plate is equal to the distance between the two peep windows provided at both ends on the visual field plate.

According to this configuration, when the single eye is trained, the shield plate is slid so that the peep windows at both the ends can be closed. When the both eyes are trained, the shield plate is located at a home position so that the peep window at the center can be closed. Thus, a more concentrated training can be realized.

Further, the vision recovery training device of the present invention is characterized in that the peep window has a form long in a transverse direction.

According to this configuration, both a child having a narrow space of pupils and an adult having a wide space of pupils can train both eyes by the same device.

### ADVANTAGE OF THE INVENTION

In the present invention, since three peep windows are provided at intervals of equal distances in a visual field plate and a distance between both ends is substantially equal to a space between pupils of a person, the vision recovery training device can be provided that can efficiently carry out both the training of the single eye and the training of the both eyes.

### BRIEF DESCRIPTION OF THE DRAWINGS

FIG. 1 shows a front view and a side view of a schematic structural diagram of a vision recovery training device in a first embodiment of the present invention.

FIG. 2 shows diagrams of carrying out a training of both eyes and a training of a single eye of the vision recovery training device in the first embodiment of the present invention.

FIG. 3 shows a schematic structural diagram main parts of a vision recovery training device in a second embodiment of the present invention.

FIG. 4 shows diagrams of carrying out a training of both eyes and a training of a single eye of the vision recovery training device in the second embodiment of the present invention.

FIG. 5 is a schematic diagram of a usual vision recovery training device.

FIG. 6 is a cross sectional view of the usual vision recovery training device.

### DESCRIPTION OF REFERENCE NUMERALS AND SIGNS

1 vision recovery training device
2 target
3 driving device
4 housing
5a, 5b, 5c peep window
6 driving direction
7 trainer
8 left eye
9 right eye
10 training mark
11 shield plate
12a, 12b hole
13 visual field plate
D diameter of peep window 5b
L width of peep windows 5a and 5c
IL inner distance between peep windows 5a and 5c
OL outer distance between peep windows 5a and 5c

### BEST MODE FOR CARRYING OUT THE INVENTION

Now, a vision recovery training device of embodiments of the present invention will be described below by referring to the drawings.

### FIRST EMBODIMENT

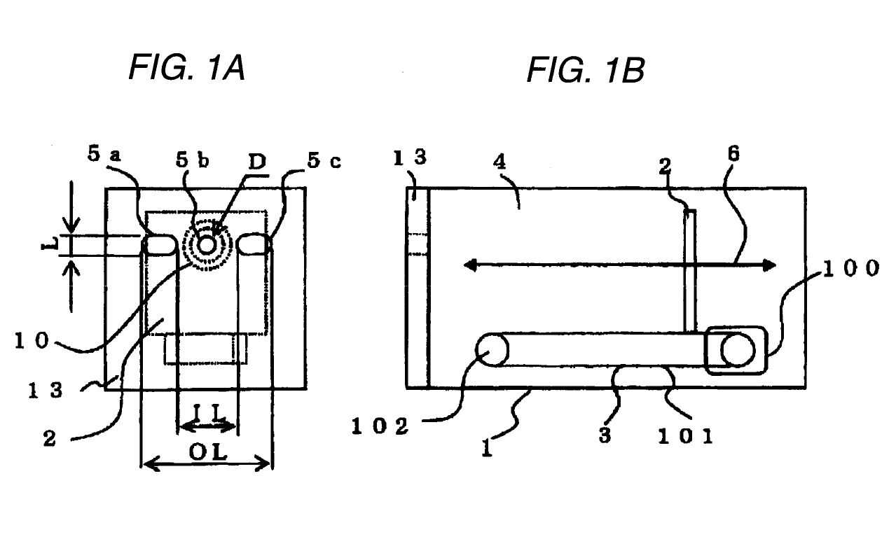

The vision recovery training device of a first embodiment of the present invention is shown in FIG. 1. FIG. 1A is a front view and FIG. 1B is a side view.

In FIG. 1, the vision recovery training device 1 includes a target 2 to be stared at, a driving device 3 for driving the target 2 and a housing 4 for internally enclosing the target 2 and the driving device 3. On the housing 4, a visual field plate 13 is disposed. On the visual field plate 13, peep windows 5a, 5b and 5c are formed. On the target 2, a training mark 10 is written. The target 2 moves in the direction shown by an arrow mark 6. The driving device 3 includes a motor 100, a belt 101 and a pulley 102.

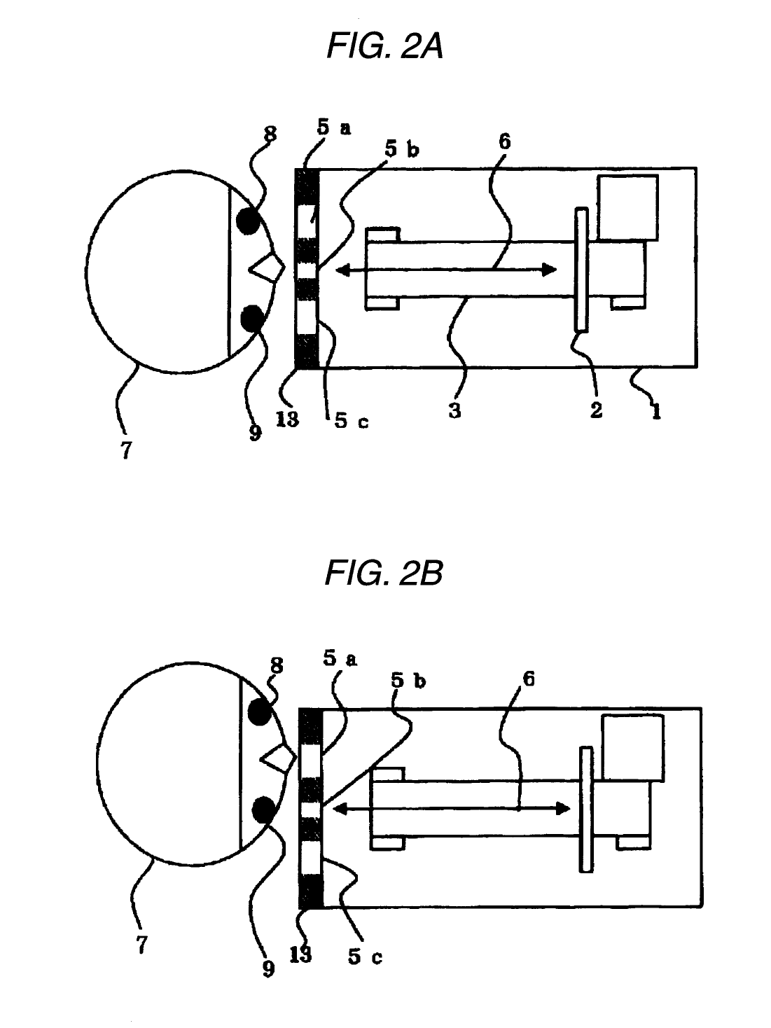

An operation of the vision recovery training device constructed as described above will be described by referring to FIG. 2.

FIG. 2A is a diagram showing a positional relation between a training device and a trainer when both eyes are trained. FIG. 2B is a diagram showing a positional relation between the training device and the trainer when a single eye is trained.

When the both eyes are trained, as shown in FIG. 2A, the trainer 7 sets a left eye 8 to the peep window 5a and sets a right eye 9 to the peep window 5c. After that, the target 2 is reciprocated in the direction shown by the arrow mark 6 and the training mark written in the reciprocating target 2 is watched by the left eye 8 and the right eye 9 so that the training by the both eyes can be carried out. On the other hand, when one eye, for instance, the right eye 9 is trained, as shown in FIG. 2B, the right eye 9 is set to the peep window 5b. At this time, the left eye 8 sees only the visual field plate 13 and cannot see the target 2. Under this state, the target 2 is reciprocated in the direction shown by the arrow mark 6 and the training mark 10 written in the reciprocating target 2 is watched by the right eye 9. Thus, a convergence does not arise in the right eye 9 and a genuine accommodative training can be carried out.

The vision recovery training device of the first embodiment of the present invention includes the target 2 to be stared at, the driving device 3 for driving the target 2, the housing 4 for internally enclosing the target 2 and the driving device 3, and the visual field plate 13 disposed on one surface of the housing 4. In the visual field plate 13, the peep windows 5a, 5b and 5c are provided so that both the training of the both eyes and the training of the single eye can be efficiently carried out by the same vision recovery training device. In FIG. 1, the peep windows 5a and 5c are formed in an oval shape. A distance between pupils of a child of 9 years old is 58 mm on an average. A distance between pupils of an adult is 62 mm on an average, and distributed from 56 mm to 72 mm. As shown in FIG. 1, assuming that an inner distance IL between the peep windows 5a and 5c is 50 mm and an outer distance OL therebetween is 80 mm, the training from the adult to the child of 9 years old or so can be carried out. When the width L of the peep windows 5a and 5c and the diameter D of the peep window 5b are made to be small enough to see the training mark 10, since other things than the training mark are hardly seen, the trainer is apt to concentrate on the training.

### SECOND EMBODIMENT

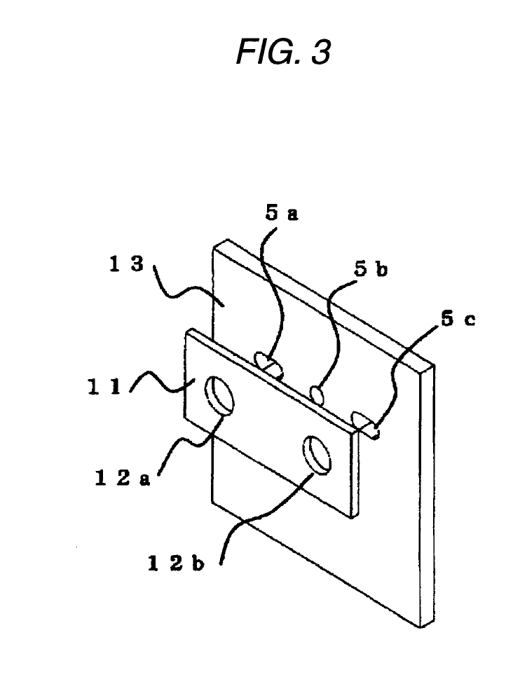

Next, main parts of a vision recovery training device of a second embodiment of the present invention is shown in FIG. 3.

In FIG. 3, a shield plate 11 is held to slide so as to cover peep windows 5a to 5c formed on a visual field plate 13 therewith. On the shield plate 11, holes 12a and 12b are formed. In this explanatory view, the shield plate 11 is shown to be separate from the visual field plate 13 so as to be easily understood, however, actually comes into contact with the visual field plate.

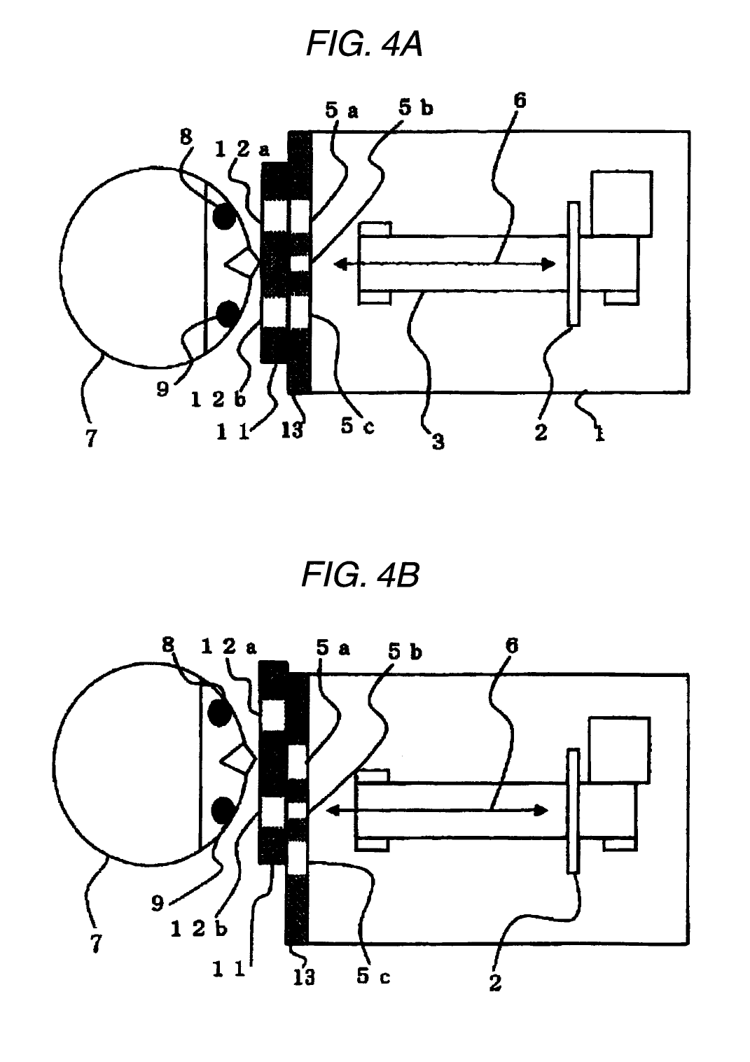

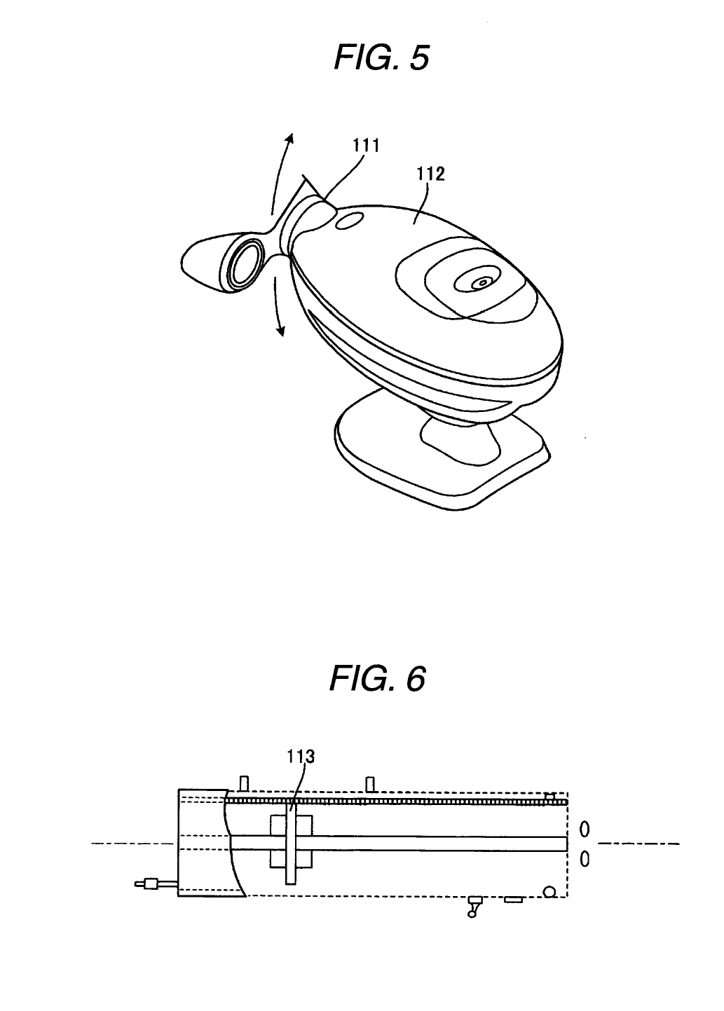

An operation of the vision recovery training device constructed as described above will be described by referring to FIG. 4. In FIG. 4, the shield plate 11 and the visual field plate 13 are shown in cross sectional views so as to be easily understood.

FIG. 4A shows a case that both eyes are trained and the hole 12a provided in the shield plate 11 corresponds to the peep window 5a and the hole 12b corresponds to the peep window 5c respectively. Accordingly, a left eye 8 and a right eye 9 can respectively watch a training mark 10 written in a target 2.

FIG. 4B shows a case that a single eye is trained, for instance, the right eye 9 is trained. The shield plate 11 is moved upward in FIG. 4B and the hole 12a of the shield plate 11 does not correspond to any of the peep windows 5a to 5c. On the other hand, the hole 12b corresponds to the peep window 5b so that the right eye 9 can watch the training mark 10 of the target 2. Further, since the right eye 9 is located on a line of the moving direction of the target, only an accommodative ability can be trained without generating a convergence. At this time, since the left eye 8 whose training is not carried out is closed, an excessive disturbance does not enter the left eye 8. Further, a trainer simply peeps at the holes 12a and 12b formed on the shield plate 11 by the both eyes so that the trainer can train the single eye without being puzzled. Further, when the shield plate 11 is located at a position for training the both eyes, a position for training the left eye and a position for training the right eye, the shield plate 11 may be pressed with a sense of click by a leaf spring or a plunger so that the shield plate 11 is not shifted.

As described above, according to the vision recovery training device of the second embodiment of the present invention, the shield plate is held to slide so as to cover the peep windows formed on a surface of a housing therewith and the two holes are formed on the shield plate. In such a configuration, both the training of the both eyes and the training of the single eye can be carried out without being puzzled. Further, during the training of the single eye, since the disturbance can be prevented from entering the single eye that is not trained, the single eye can be more efficiently trained.

The present invention is described in detail by referring to the specific embodiments, however, it is to be understood for a person with ordinary skill in the art that various changes or modifications can be made without departing from the spirit and scope of the present invention.

This application is based on Japanese Patent Application (No. 2005-284152) filed on Sep. 29, 2005 and its contents are incorporated herein as a reference.

### INDUSTRIAL APPLICABILITY

As described above, since in the vision recovery training device according to the present invention, the three peep windows are provided at intervals of equal distances in the visual field and a distance between both ends is substantially equal to a space between pupils of a person, the vision recovery training device can be effectively provided that can efficiently carry out both the training of the single eye and the training of the both eyes. The vision recovery training device is useful as a vision recovery training device for training an image formation accommodative function of an eye.

1. A vision recovery training device for training an image formation accommodative function of an eye, comprising:
a target to be watched;
a driving device that moves the target; and
a visual field plate having three peep windows through which the target is peeped,
wherein the three peep windows are provided and respectively horizontally arranged in parallel;
wherein the peep window provided at a center is located on an extended line of a moving direction of the target;
wherein other two peep windows are located at positions spaced by an equal distance from the peep window located at the center; and
wherein a distance between the other two peep windows is substantially equal to an interval of pupils of a person.

2. The vision recovery training device according to claim 1, further comprising a shield plate that covers the visual field plate and is held so as to slide,
wherein two holes are formed on the shield plate; and
wherein a distance between the two holes formed on the shield plate is equal to the distance between the two peep windows provided at both ends on the visual field plate.

3. The vision recovery training device according to claim 1, wherein each of the peep windows has a form long in a transverse direction.
